(19) United States
(12) Patent Application Publication
(10) Pub. No.: US 2026/0014203 A1
(43) Pub. Date: Jan. 15, 2026

(54) USE OF COMPOSITION INCLUDING MESENCHYMAL STEM CELLS FOR ALLEVIATING MYELOFIBROSIS

(71) Applicant: MEDICAL AND PHARMACEUTICAL INDUSTRY TECHNOLOGY AND DEVELOPMENT CENTER, New Taipei City (TW)

(72) Inventors: Hsiu-Hsia LIN, New Taipei City (TW); Hung-Jun LIN, New Taipei City (TW); Ping-Lun JIANG, New Taipei City (TW); Chung-Pin HSIEH, New Taipei City (TW); Yi-Chih KUO, New Taipei City (TW); Chien-Yu KAO, New Taipei City (TW)

(21) Appl. No.: 19/107,452

(22) PCT Filed: Aug. 30, 2023
(86) PCT No.: PCT/CN2023/115740

§ 371 Date: Feb. 27, 2025

Related U.S. Application Data
(60) Provisional application No. 63/374,386, filed on Sep. 2, 2022.

(51) Int. Cl. A61K 35/28

(52) U.S. Cl. CPC A61K 35/28

(57) ABSTRACT

A method for alleviating myelofibrosis includes administering to a subject in need thereof a composition including mesenchymal stem cells (MSCs). The MSCs may be selected from umbilical cord-derived mesenchymal stem cells (UCMSCs), bone marrow-derived mesenchymal stem cells (BMMSCs), adipose tissue-derived mesenchymal stem cells (AMSCs), dermis-derived mesenchymal stem cells (DMSCs), epidermis-derived mesenchymal stem cells (EMSCs), synovial membrane-derived mesenchymal stem cells (SMMSCs), dental tissue-derived mesenchymal stem cells (dental MSCs), lung-derived mesenchymal stem cells (LMSCs), and combinations thereof.

### CROSS-REFERENCE TO RELATED APPLICATION

This application is a national phase entry application of PCT International Application No. PCT/CN2023/115740, filed on Aug. 30, 2023, which claims priority of U.S. Provisional Patent Application No. 63/374,386, filed on Sep. 2, 2022. The aforesaid applications are incorporated by reference herein in their entirety.

### FIELD

The present disclosure relates to use of a composition including mesenchymal stem cells for alleviating myelofibrosis.

### SEQUENCE LISTING XML

The Sequence Listing submitted concurrently herewith with a file name of “PE-68332-WO-SEQUENCE LISTING.xml,” a creation date of Aug. 24, 2023, and a size of 20.4 kilobytes, is part of the specification and is incorporated by reference in its entirety.

### BACKGROUND

Myelofibrosis is a type of myeloproliferative neoplasms (MPNs), a group of cancers in which there is growth of abnormal cells in the bone marrow. Myelofibrosis is often associated with BCR-ABL1 fusion negative and overactivation of JAK-dependent signaling caused by JAK2, MPL and CALR mutations. In myelofibrosis, healthy marrow is replaced by scar tissue (fibrosis), resulting in cytopenia, which leads to anemia, increased susceptibility to infection, and splenomegaly, among others. Myelofibrosis can be divided into primary myelofibrosis and secondary myelofibrosis based on pathogenic progression. Primary myelofibrosis (PMF) is myelofibrosis that occurs without any preceding MPNs, whereas secondary myelofibrosis refers to bone marrow fibrosis that develops from other MPNs, such as essential thrombocythemia (ET) and polycythemia vera (PV), and can be classified as post-ET MF and post-PV MF.

The only curative therapy for myelofibrosis is allogeneic hematopoietic stem-cell transplantation (HSCT), in which patients undergo complete or partial bone marrow ablation (i.e., myeloablative or nonmyeloablative HSCT) to remove mutant MPN and stromal cells in bone marrow and then receive hematopoietic stem cells from healthy donor; however, allogenic HSCT is only utilized in 5% of patients because of human leukocyte antigen matching requirement and severe complications, including graft-versus-host disease (GVHD). Till date, the U.S. Food and Drug Administration (FDA) has approved three JAK inhibitors, i.e., ruxolitinib (Jakavi), fedratinib (Inrebic) and pacritinib (Vonjo), in treatment of intermediate or high-risk myelofibrosis; however, these drugs merely alleviate splenomegaly and some of the constitutional symptoms without improving anemia and bone marrow fibrosis.

In myelofibrosis, fibrosis of bone marrow stromal cells could be induced by TGF-β1, megakaryocyte, or MPN cells. Inflammatory cytokines, such as IL-1β, IL-6, and TNF-α, which are released from fibrotic bone marrow stromal cells, megakaryocytes or MPN cells, are critical in the pathogenic progression of myelofibrosis. Targeting to TGF-β1, megakaryocytes, MPN cells, and such inflammatory cytokines have been reported to alleviate myelofibrosis by reducing bone marrow fibrosis, and/or indirectly regulate megakaryocytes and MPN cells differentiation and proliferation.

Mesenchymal stem cells (MSCs), after being phagocytosed, have been reported to possess immunomodulatory property, and thus can effectively reduce inflammatory cytokines. In recent years, several studies have reported the use of MSCs in HSCT to control GVHD in patients diagnosed with myelofibrosis; however, the efficiency of this approach remains unclear due to limited number of cases and no improvement in the patients' lifespan after HSCT.

Therefore, there is an urgent need to develop a new method for effective treatment of myelofibrosis.

### SUMMARY

Therefore, an object of the present disclosure is to provide a method for alleviating myelofibrosis which can alleviate at least one of the drawbacks of the prior art.

According to the present disclosure, the method includes administering to a subject in need thereof a composition including mesenchymal stem cells.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 shows the platelet number (left panel), red blood cells (RBC) number (middle panel) and hemoglobin concentration (right panel) in the TPO-overexpressing mice at the 8^{th} week post-transplantation of Example 1, infra, in which the symbols “*” and “***” respectively represent p<0.05 and p<0.001 compared with the control.

FIG. 2 shows (a) the hemoglobin concentration (left panel) and RBC number (right panel) in the mice of each group before and after administration of umbilical cord-derived mesenchymal stem cells (UCMSCs), (b) and (c) size and weight of spleen of the mice in each group, (d) hematoxylin and eosin (H & E) staining of bone marrow of the mice in each group, (e) Periodic acid-Schiff (PAS) staining of bone marrow of the mice in each group, and (f) relative percentage of PAS-stained (PAS⁺) area (left panel) and relative percentage of PAS⁺ intensity (right panel) of the bone marrow of the mice in each group of Example 1, infra.

FIG. 3 shows the relative mRNA expression levels of (a) COL1A1 gene, (b) FN1 gene, (c) ACTA2 gene, (d) IL-1β gene, (e) TNF-α gene, and (f) TGF-β1 gene in the bone marrow stromal cells of each group of Example 2, infra, in which the symbol “***” represents p<0.001 compared with the normal control group, the symbols “#”, “##” and “###” respectively represent p<0.05, p<0.01 and p<0.001 compared with the pathological control group.

FIG. 4 shows the relative mRNA expression levels of (a) COL1A1 gene, (b) FN1 gene, (c) ACTA2 gene, (d) IL-1β gene, (e) TNF-α gene, and (f) TGF-β1 gene in the bone marrow stromal cells of each group of Example 2, infra, in which the symbols “*”, “**” and “***” respectively represent p<0.05, p<0.01 and p<0.001 compared with the normal control group, and the symbols “#” and “###” respectively represent p<0.05 and p<0.001 compared with the pathological control group 3.

FIG. 5 shows the relative mRNA expression levels of (a) COL1A1 gene, (b) FN1 gene, (c) ACTA2 gene, (d) IL-1β gene, and (e) IL-6 gene in the bone marrow stromal cells of each group of Example 2, infra.

FIG. 6 shows the relative mRNA expression levels of (a) COL1A1 gene, (b) FN1 gene, (c) ACTA2 gene, (d) IL-1β gene, and (e) IL-6 gene in the bone marrow stromal cells of each group of Example 2, infra, in which the symbols “*” and “**” respectively represent p<0.05 and p<0.01 compared with the normal control group, and the symbols “#” and “##” respectively represent p<0.05 and p<0.01 compared with the pathological control group 2.

FIG. 7 shows the relative mRNA expression levels of (a) IL-1β gene, (b) IL-6 gene, (c) TNF-α gene, and (d) TGF-β1 gene in the MEG-01 cells of each group of Example 2, infra.

FIG. 8 shows the relative mRNA expression levels of (a) IL-6 gene and (b) TNF-α gene in the HEL cells of each group of Example 2, infra.

FIG. 9 shows the relative mRNA expression levels of (a) COL1A1 gene and (b) FN1 gene, in the bone marrow stromal cells of each group of Example 2, infra, in which the symbols “**” and “***” respectively represent p<0.01 and p<0.001 compared with the normal control group, and the symbols “#” and “###” respectively represent p<0.05 and p<0.001 compared with the pathological control group.

FIG. 10 shows the relative mRNA expression levels of (a) COL1A1 gene and (b) FN1 gene, in the bone marrow stromal cells of each group of Example 2, infra, in which the symbol “***” represents p<0.001 compared with the normal control group, and the symbols “##” and “###” respectively represent p<0.01 and p<0.001 compared with the pathological control group.

FIG. 11 shows the percentage of indoleamine-pyrrole 2,3-dioxygenase (IDO)-expressing UCMSCs in each group after treatment with different concentrations of IFN-γ of Example 3, infra.

FIG. 12 shows the percentage of IDO-expressing UCMSCs in each group after treatment with different concentrations of IFN-γ for different time periods of Example 3, infra.

FIG. 13 shows the relative mRNA expression level of IDO gene in each group after treatment with IFN-γ in combination with a supplement of Example 3, infra.

FIG. 14 shows the relative percentage of IDO-expressing UCMSCs in each group after treatment with IFN-γ for different time period in combination with budesonide for different time period of Example 3, infra.

FIG. 15 shows the relative mRNA expression levels of (a) COL1A1 gene, (b) FN1 gene and (c) ACTA2 gene in the bone marrow stromal cells of each group of Example 3, infra, in which the symbol “***” represents p<0.001 compared with the normal control group, the symbols “#”, “##” and “###” respectively represent p<0.05, p<0.01 and p<0.001 compared with the pathological control group, and the symbols “$$” and “$$$” respectively represent p<0.01 and p<0.001 compared with the comparative group 1.

FIG. 16 shows the relative mRNA expression levels of (a) COL1A1 gene, (b) FN1 gene and (c) ACTA2 gene in the bone marrow stromal cells of each group of Example 3, infra, in which the symbols “*”, “**” and “***” respectively represent p<0.05, p<0.01, and p<0.001 compared with the normal control group, the symbols “#”, “##” and “###” respectively represent p<0.05, p<0.01 and p<0.001 compared with the pathological control group, and the symbol “$$” represents p<0.01 compared with the comparative group.

### DETAILED DESCRIPTION

Before the present disclosure is described in greater detail, it should be noted that if any prior art publication is referred to herein, such reference does not constitute an admission that the publication forms a part of the common general knowledge in the art.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In order to address the current limitations of JAK inhibitors in the treatment of myelofibrosis, the applicants endeavored to develop improved methods and found that administration of mesenchymal stem cells (MSCs) to a myelofibrosis mouse model are capable of alleviating the three major clinical symptoms of myelofibrosis, i.e., anemia, splenomegaly and bone marrow fibrosis. In addition, in vitro experiments showed that MSCs exhibited anti-fibrosis and anti-inflammatory effects in bone marrow stromal cells, megakaryocytes, and MPN cells. Furthermore, primed MSCs, which are formed by subjecting MSCs to priming, in comparison with normal MSCs (i.e., non-primed MSCs), demonstrated greater efficacy against fibrosis in the bone marrow stromal cells.

The present disclosure provides a method for alleviating myelofibrosis which includes administering to a subject in need thereof a composition including mesenchymal stem cells (MSCs).

The present disclosure also provides use of the aforesaid composition in the manufacture of a medicament for alleviating myelofibrosis in a subject.

Examples of the MSCs include, but are not limited to, umbilical cord-derived mesenchymal stem cells (UCMSCs), bone marrow-derived mesenchymal stem cells (BMMSCs), adipose tissue-derived mesenchymal stem cells (AMSCs), dermis-derived mesenchymal stem cells (DMSCs), epidermis-derived mesenchymal stem cells (EMSCs), synovial membrane-derived mesenchymal stem cells (SMMSCs), dental tissue-derived mesenchymal stem cells (dental MSCs), and lung-derived mesenchymal stem cells (LMSCs). In certain embodiments, the MSCs are UCMSCs.

In certain embodiments, the UCMSCs may be non-primed UCMSCs which are not subjected to priming. In certain embodiments, the UCMSCs may be primed UCMSCs which have been subjected to priming. In an exemplary embodiment, the primed UCMSCs are indoleamine-pyrrole 2,3-dioxygenase (IDO)-expressing UCMSCs.

According to the present disclosure, the IDO-expressing UCMSCs may be prepared by cultivation of the non-primed UCMSCs in a culture medium supplemented with IFN-γ for a time period ranging from 24 hours to 72 hours. In an exemplary embodiment, the IDO-expressing UCMSCs may be prepared by cultivation of the non-primed UCMSCs in a culture medium supplemented with IFN-γ for 72 hours.

In certain embodiments, in addition to the IFN-γ, the culture medium is further supplemented with a substance. Examples of the substance may include, but not limited to, vitamin, steroid, cytokine, double-stranded RNA, and histone deacetylase (HDAC) inhibitor. In an exemplary embodiment, the vitamin is retinoic acid, the steroid is dexamethasone and budesonide, the cytokine is TNF-α, the double-stranded RNA is polyinosinic acid-polycytidylic acid, and the HDAC inhibitor is valproic acid.

As used herein, the term “subject” refers to any animal of interest, such as humans, monkeys, cows, sheep, horses, pigs, goats, dogs, cats, mice, and rats. In certain embodiments, the subject is a mice. In certain embodiments, the subject is a human.

In certain embodiments, the subject is not subjected to myeloablative transplantation.

As used herein, the term “administration” or “administering” means introducing, providing or delivering a pre-determined active ingredient to a subject by any suitable routes to perform its intended function.

Examples of the myelofibrosis may include, but not limited to, primary myelofibrosis, post-essential thrombocythemia myelofibrosis, and post-polycythemia vera myelofibrosis.

According to the present disclosure, the composition may be formulated into a dosage form suitable for parenteral administration using technology well known to those skilled in the art.

According to the present disclosure, for parenteral administration, the composition according to the present disclosure may be formulated into an injection, e.g., a sterile aqueous solution, a dispersion or an emulsion.

The composition according to the present disclosure may be administered via one of the following parenteral routes: intraperitoneal injection, intrapleural injection, intramuscular injection, intravenous injection, intracarotid injection, intraarterial injection, intraarticular injection, intrasynovial injection, intrathecal injection, intracranial injection, intraepidermal injection, subcutaneous injection, intradermal injection, and intralesional injection.

In certain embodiments, the composition is administered by intravenous injection.

In certain embodiments, after administration of the composition, the subject shows reduced level of bone marrow fibrosis and ameliorated inflammation.

In certain embodiments, after administration of the composition, the subject further shows ameliorated splenomegaly and ameliorated anemia.

The dose and frequency of administration of the composition may vary depending on the following factors: the severity of the illness or disorder to be treated, routes of administration, and age, physical condition and response of the subject to be treated. In general, the composition may be administered in a single dose or in several doses.

The present disclosure will be described by way of the following examples. However, it should be understood that the following examples are intended solely for the purpose of illustration and should not be construed as limiting the present disclosure in practice.

### EXAMPLES

### General Experimental Materials:

### 1. Source and Cultivation of Human Bone Marrow Stromal Cells, Human Erythroleukemia (HEL) Cells, Megakaryoblastic MEG-01 Cells, and Megakaryoblastic SET-2 Cells

Human bone marrow stromal cells, also known as bone marrow-derived mesenchymal stem cells (BMMSCs), were purchased from ScienCell Research Laboratories, HEL 92.1.7 cell line and MEG-01 cell line were obtained from American Type Culture Collection, and SET-2 cell line was obtained from Deutsche Sammlung von Mikroorganismen und Zellkulturen.

Human bone marrow stromal cells were cultured in α-MEM medium (Gibco) supplemented with 5% human platelet lysate (HPL, purchased from Sexton Biotechnologies). Each of the HEL 92.1.7 cells and MEG-01 cells were cultivated in RPMI 1640 medium (Gibco) supplemented with 10% fetal bovine serum (FBS) (Gibco), and the SET-2 cells were cultivated in RPMI 1640 medium supplemented with 20% FBS.

### 2. Source and Cultivation of Umbilical Cord-Derived Mesenchymal Stem Cells (UCMSCs)

UCMSCs were isolated from human umbilical cord tissues according to procedures that were modified from the procedures described by Mori Y. et al., in an article entitled “Improved explant method to isolate umbilical cord-derived mesenchymal stem cells and their immunosuppressive properties” published in Tissue Eng Part C Methods, 2015, Vol. 21, p. 367-372. In brief, human umbilical cord tissues were immersed in cold phosphate-buffered saline (PBS) and then cut into tissue fragments. The middle portion of the umbilical cord tissue fragments was cut to expose Wharton's jelly, followed by placement of the Wharton's jelly into 10-cm dishes. Next, α-MEM medium supplemented with 10% HPL were immediately added into the dishes. The resultant explant was incubated at 37° C., 5% CO₂ for 7 days after plating, followed by removal of the umbilical cord tissue fragments and replacement of the culture medium (i.e., α-MEM medium supplemented with 5% HPL) every 2 days until the fibroblast-like adherent UCMSCs cells reached 80% to 90% confluence. The UCMSCs migrating from the explants and the tissue fragments were collected using TrypLE™ (Gibco) within 10 to 14 days after plating.

Afterwards, UCMSCs culture was prepared by seeding the UCMSCs at 3000 to 5000 cells/cm² per culture dish into respective culture dishes containing α-MEM medium (Gibco) supplemented with 5% HPL (Sexton Biotechnologies). Culture medium replacement was conducted every 2 days, and the proliferated UCMSCs were subjected to cryopreservation in NutriFreez® D10 Cryopreservation Medium (Manufacturer: Corning).

### 3. Experimental Animals

The experimental animals, i.e., C57BL/6 mice, used in the following experiments were purchased from BioLASCO. All the experimental animals were housed in an animal room with an independent air conditioning system under the following laboratory conditions: an alternating 12-hour light and 12-hour dark cycle, a temperature maintained at 23° C.±2° C. and a relative humidity maintained at 50%±10%. The experimental animals were provided with water and fed ad libitum. All experimental procedures involving the experimental animals were in compliance with the legal provision of the Animal Protection Act of Taiwan, China, and were carried out according to the guidelines of the Animal Care Committee of the Council of Agriculture, Taiwan, China.

### General Experimental Procedures:

### 1. Statistical Analysis

All the experiments described below were performed at least 3 times. The experimental data of all the test groups are expressed as mean±standard error of the mean (SEM), and were analyzed using two-tailed Student's t-test or one-way ANOVA followed by Newman-Keuls post hoc test using Prism 6.0 software (Developer: GraphPad software), so as to assess the differences between the groups. Statistical significance is indicated by p<0.05.

### Example 1. Preparation of TPO-Overexpressing Mice and Evaluation of the Effect of Umbilical Cord-Derived Mesenchymal Stem Cells (UCMSCs) on Alleviation of Myelofibrosis in TPO-Overexpressing Mice

In this example, TPO (thrombopoietin)-overexpressing mice were prepared and subjected to validation of induction of myelofibrosis, followed by evaluation of the effect of UCMSCs on alleviation of myelofibrosis in the TPO-overexpressing mice.

### A. Preparation of TPO-Overexpressing Mice

TPO-overexpressing mice were prepared according to the procedures described by Yan X. Q. et al., in an article entitled “A model of myelofibrosis and osteosclerosis in mice induced by overexpressing thrombopoietin (mpl ligand): reversal of disease by bone marrow transplantation” published in Blood, 1996, Vol. 88, p. 402-409. In brief, C57BL/6 mice (6 to 8 weeks old) (n=6) were administered, via intraperitoneal injection, with 5-fluorouracil at a dose of 150 mg/kg. On the fourth day post-administration, bone marrow tissue were extracted from the mice and then incubated with red blood cells (RBC) lysis buffer for 3 minutes. Next, bone marrow cells were seeded at a concentration of 5×10⁶ cells per well into respective wells of 12-well plates each containing 1 mL of StemSpan SFEM II medium (STEMCELL Technologies Inc.) supplemented with 50 ng/mL of mouse stem cell factor (SCF), 50 ng/mL of human TPO, 20 ng/mL of mouse IL-3, and 50 ng/mL of mouse Flt-3 ligand, and were immediately subjected to a first infection with retrovirus containing TPO cDNA (Manufacturer: Cell Biolabs; Catalog no. RV-101) according to the manufacturer's protocol, followed by a second infection at the 24^{th} hour after the first infection. At the 16^{th} hour after the second infection, 5×10⁶ floating cells were collected as donor cells and 2×10⁵ cells of such donor cells were implanted, via intraorbital injection, into recipient C57BL/6 mice which were earlier subjected to γ-irradiation with caesium-137 at a dose of 17 Gy, followed by feeding the recipient mice with water containing 1 mM neomycin for 2 weeks, so as obtain TPO-overexpressing mice.

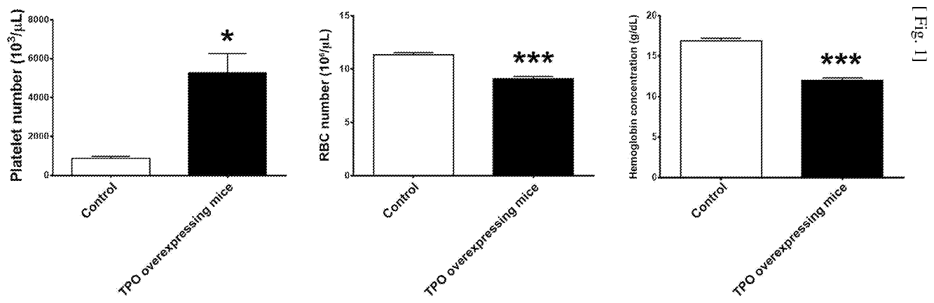

At the 8^{th} week post-transplantation, the TPO-overexpressing mice were subjected to determination of hematopoiesis effects by measuring the levels of platelet, RBC and hemoglobin to be compared with those of healthy C57BL/6 mice (serving as control), so as to validate the induction of myelofibrosis in the TPO-overexpressing mice. In brief, blood was collected from the retro-orbital plexus of each mouse and subjected to measurement of platelet and red blood cells (RBC) counts, and hemoglobin concentration using ProCyte Dx Hematology Analyzer. The results are shown in FIG. 1.

FIG. 1 shows the platelet number, RBC number and hemoglobin concentration in the TPO-overexpressing mice and the control at the 8^{th} week post-transplantation. As shown in FIG. 1, in comparison to the control, the TPO-overexpressing mice showed significantly increased number of platelets, and significantly reduced number of RBC and hemoglobin concentration, indicating that myelofibrosis was successfully induced in the TPO-overexpressing mice.

### B. Evaluation of Effect of UCMSCs on Alleviation of Myelofibrosis

Cryopreserved UCMSCs were thawed in water bath at 37° C. and the thawed UCMSCs were then immersed in α-MEM medium (Gibco) supplemented with 5% HPL (Sexton Biotechnologies), followed by centrifugation at 350 g for 5 minutes and washing in cold PBS.

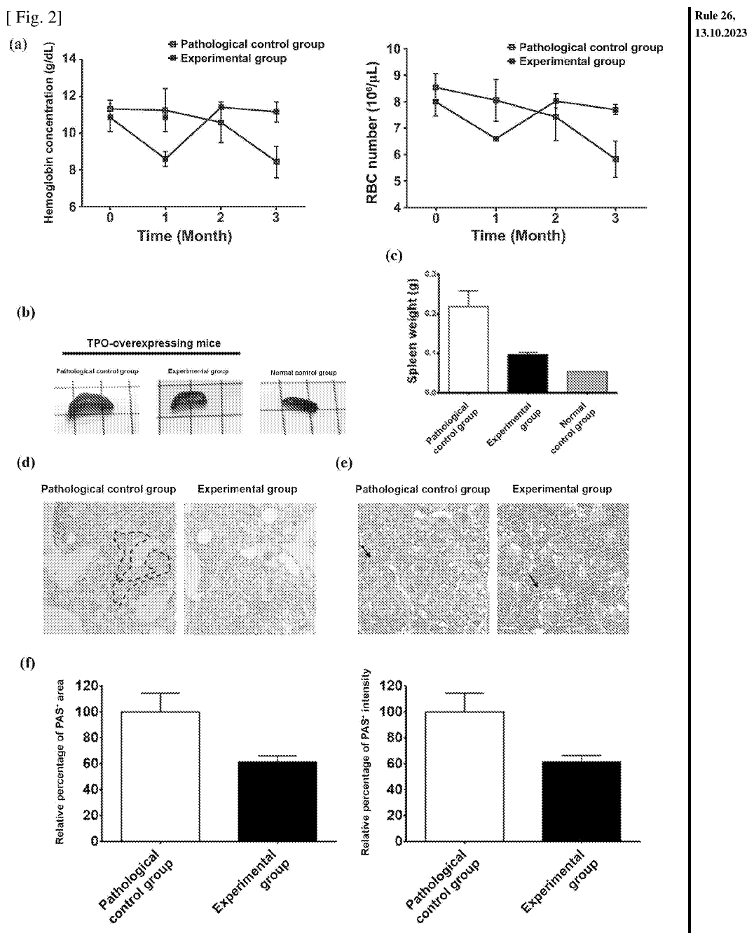

The TPO-overexpressing mice that was validated to have myelofibrosis, as described in section A above, were divided into two groups, i.e., a pathological control group (PCG) and an experimental group (EG), with n=3 in each group. Healthy C57BL/6 mice (n=3), which were not subjected to transplantation, served as a normal control group (NCG). The mice in the EG were administered, via intravenous injection at a rate of 100 μL/minute into the tail vein, with 5×10⁵ UCMSCs at the 1^{st}, 2^{nd}, 4^{th} and 5^{th} week after validation of myelofibrosis (i.e., 0 month), while the mice in the NCG were administrated with PBS. In addition, the mice in the PCG received no treatment. At one, two and three months after administration of USMSCs, blood was collected from the mice in the PCG and EG for measurement of hemoglobin concentration and RBC number. Following the blood collection at three months after administration of UCMSCs, the mice in the NCG, PCG and EG were sacrificed, and then subjected to observation of spleen size and measurement of spleen weight. Moreover, the bone marrows of the mice in the PCG and EG were subjected to identification of bone marrow fibrosis using hematoxylin and eosin (H&E) staining and Periodic acid-Schiff (PAS) staining that could stain carbohydrates in different types of collagen and that was conducted according to the procedures described by Steinke H. et al., in an article entitled “Periodic acid-Schiff (PAS) reaction and plastination in whole body slices. A novel technique to identify fascial tissue structures” published in Ann Annat, 2018, Vol. 216, p. 29-35, and were further subjected to grading of bone marrow fibrosis according to the procedures described by Thiele J. et al., in an article entitled “European consensus on grading bone marrow fibrosis and assessment of cellularity” published in Haematologica, 2005, Vol. 90, p. 1128-1132. The results are shown in FIG. 2(a) to (f).

FIG. 2(a) shows the hemoglobin concentration (left panel) and RBC number (right panel) in the mice of each group at the 0 month (i.e., at the 8^{th} week post-transplantation), and at 1 month, 2 months and 3 months after administration of UCMSCs. As shown in FIG. 2(a), starting from the 0 month to 3 months after administration of the UCMSCs, the mice in the pathological control group showed gradual reduction in hemoglobin concentration and RBC number, whereas the mice in the experimental group showed increase in the hemoglobin concentration and RBC number 2 months after administration of the UCMSCs, indicating that the TPO-overexpressing mice showed ameliorated anemia after treatment with UCMSCs.

FIGS. 2(b) and (c) respectively show the size and weight of the spleen of the mice in each group 3 months after administration of the UCMSCs. As shown in FIGS. 2(b) and (c), the mice in the pathological control group have spleen that is larger in size and greater in weight compared to those of the normal control group, while the mice in the experimental group showed reduced size and weight of the spleen compared to those of the pathological control group, indicating that the TPO-overexpressing mice showed ameliorated splenomegaly after treatment with UCMCSs.

FIGS. 2(d) and (e) respectively show hematoxylin and eosin (H & E) staining and Periodic acid-Schiff (PAS) staining of bone marrow of the mice in each group 3 months after administration of the UCMSCs. As shown in FIGS. 2(d) and (e), atypical cell streaming was observed in the pathological control group (indicated by the circled area) but was absent in the experimental group, while the number of PAS-stained (PAS⁺) cells (indicated by the arrow) was greater in the pathological control group compared to the experimental group.

FIG. 2(f) shows the relative percentage of PAS⁺ area (left panel) and relative percentage of PAS⁺ intensity (right panel) of the bone marrow of the mice in each group 3 months after administration of the UCMSCs. As shown in FIG. 2(f), in comparison with the pathological control group, the relative percentage of PAS⁺ area and relative percentage of PAS⁺ intensity in the experimental group were reduced to 61.34±4.93% and 61.45±4.97%, respectively, indicating that the TPO-overexpressing mice showed reduced level of bone marrow fibrosis after treatment with UCMCSs.

These results demonstrated that UCMSCs are capable of alleviating the three major clinical symptoms of myelofibrosis, i.e., anemia, splenomegaly and bone marrow fibrosis, in a myelofibrosis mouse model.

### Example 2. Evaluation of Effect of UCMSCs on Alleviation of Myelofibrosis Induced by Various Factors

In this example, human bone marrow stromal cells and/or myeloproliferative neoplasms (MPN) cells were used to establish cellular model of myelofibrosis, followed by determining the effect of UCMSCs on such cellular model.

### A. Effect of UCMSCs on TGF-β1-Induced Fibrosis and Inflammation in Human Bone Marrow Stromal Cells

Since TGF-β1 was reported to mediate bone marrow fibrosis in myelofibrosis, in this experiment, human bone marrow stromal cells were incubated with TGF-β1, followed by treatment with UCMSCs and determination of the expression levels of gene markers for fibrosis and inflammation.

First, the human bone marrow stromal cells were divided into 6 groups, namely, a normal control group (NCG), a pathological control group (PCG), a comparative group (CG), and 3 experimental groups, i.e., experimental groups 1 to 3 (EG1 to EG3), with the number of cells in each group being 4×10⁴ cells. Next, the human bone marrow stromal cells in the PCG and EG1 to EG3 were subjected to induction of fibrosis and inflammatory response by incubation with 5 ng/mL of TGF-β1 for 72 hours, while the human bone marrow stromal cells in the NCG and CG were not subjected to treatment with TGF-β1. Afterwards, the human bone marrow stromal cells in the CG, EG1, EG2 and EG3 were subjected to treatment with UCMSC at a ratio of cell number of UCMSCs to human bone marrow stromal cells of 1:1, 1:4, 1:2 and 1:1, respectively, for 72 hours, while the human bone marrow stromal cells in the NCG and PCG received no treatment. Subsequently, the human bone marrow stromal cells in each group were subjected to determination of relative mRNA expression levels of marker genes for fibrosis, i.e., collagen type 1 alpha 1 chain (COL1A1), fibronectin 1 (FN1), and actin alpha 2 (ACTA2), and also to determination of relative mRNA expression levels of marker genes for inflammation, i.e., IL-1β, TNF-α, TGF-β1, and IL-6. Please note that, in this experiment, the relative mRNA expression level of IL-6 in the human bone marrow stromal cells of each group was not determined.

To be specific, after removal of the UCMSCs, the human bone marrow stromal cells in each group were subjected to total RNA extraction using RNeasy Mini Kit (QIAGEN) in accordance with the manufacturer's instruction. The resultant RNA of the respective group was used as a template for synthesizing cDNA by reverse transcription polymerase chain reaction (RT-PCR) using GScript First-Strand Synthesis Kit (GeneDirex) and FastStart Essential DNA Green Master (Roche). The thus obtained cDNA, serving as a DNA template, was subjected to quantitative real-time PCR based on SYBR-Green I fluorescence, which was performed on a LightCycler® 96 System (Roche) using a designed primer pair specific for each of the 6 marker genes, i.e., COL1A1 gene, FN1 gene, ACTA2 gene, IL-1β gene, TNF-α gene, and TGF-β1 gene shown in Table 1 and the reaction conditions shown in Table 2. Peptidylprolyl isomerase A (PPIA) gene was used as an endogenous control in the quantitative analysis of real-time PCR to normalize the gene expression data.

TABLE 1

| | | | Size of |
|---|---|---|---|
| | | | PCR |
| | | Nucleotide sequence | product |
| Marker gene | PCR primer | (5′->3′) | (bp) |
| COL1A1 gene | Forward primer | tcgaggattgccoggaacag | 233 |
| (GenBank accession | COL1A1-F | (SEQ ID NO. 1) | |
| no. XM_054315082) | Reverse primer | caccagtagcaccatcatttccac | |
| | COL1A1-R | (SEQ ID NO. 2) | |
| FN1 gene | Forward primer | cacctotgtgcagaccacat | 244 |
| (GenBank accession | FN1-F | (SEQ ID NO. 3) | |
| no. NM_001365522) | Reverse primer | agttgccaccgtaagtctgg | |
| | FN1-R | (SEQ ID NO. 4) | |
| ACTA2 gene | Forward primer | agcgtggctattccttcgtt | 153 |
| (GenBank accession | ACTA2-F | (SEQ ID NO. 5) | |
| no. NM_001406462) | Reverse primer | gcccatcaggcaactogtaa | |
| | ACTA2-R | (SEQ ID NO. 6) | |
| IL-1β | Forward primer | gaccaccactacagcaaggg | 189 |
| (GenBank accession | IL-1β-F | (SEQ ID NO. 7) | |
| no. NM_000576) | Reverse primer | atcgtgcacataagoctcgt | |
| | IL-1β-R | (SEQ ID NO. 8) | |
| TNF-α | Forward primer | cactttggagtgatoggccc | 129 |
| (GenBank accession | TNF-α-F | (SEQ ID NO. 9) | |
| no. NM_000594) | Reverse primer | togtacaacatgggctacagg | |
| | TNF-α-R | (SEQ ID NO. 10) | |
| TGF-β1 | Forward primer | tggtggaaacccacaacgaa | 113 |
| (GenBank accession | TGF-β1-F | (SEQ ID NO. 11) | |
| no. NM_000660) | Reverse primer | gagcaacacgggttcaggta | |
| | TGF-β1-R | (SEQ ID NO. 12) | |
| IL-6 | Forward primer | gacagccactcacctottca | 124 |
| (GenBank accession | IL-6-F | (SEQ ID NO. 13) | |
| no. NM_001371096) | Reverse primer | agtgcctctttgctgctttc | |
| | IL-6-R | (SEQ ID NO. 14) | |
| PPIA gene | Forward primer | cgcgtctcctttgagctgtt | 175 |
| (GenBank accession | PPIA-F | (SEQ ID NO. 15) | |
| no. NM_021130) | Reverse primer | acttgocaccagtgccatta | |
| | PPIA-R | (SEQ ID NO. 16) | |

TABLE 2

| | Volume |
|---|---|
| Reaction mix | (μL) |
| cDNA (20 ng/μL) | 5 |
| Forward primer (10 μM) | 1 |
| Reverse primer (10 μM) | 1 |
| FastStart Essential DNA Green Master (2 × | 10 |
| concentrated master mix) | |
| (Manufacturer: Roche; Cat. no. 06924204001) | |
| Deionized water | 3 |

Operating conditions: 45 cycles of the following reactions: denaturation at 95° C. for 3 minutes, annealing at 95° C. for 10 seconds, and extension at 60° C. for 10 seconds.

The resultant PCR product was subjected to determination of fluorescence intensity, followed by calculating the cycle threshold (Ct) value of each of COL1A1 gene, FN1 gene, ACTA2 gene, IL-1β gene, TNF-α gene, and TGF-β1 gene. Quantitative real-time PCR data were analyzed using the comparative Ct method. In brief, the Ct value of each of the marker genes (i.e., COL1A1 gene, FN1 gene, ACTA2 gene, IL-1β gene, TNF-α gene, and TGF-β1 gene) in each group was normalized with that of PPIA gene, and the relative mRNA expression level of each of the marker genes was further calculated using the following Equation (I):

$$A = {2}^{- ( B - C )} \qquad ( I )$$

where
A=relative mRNA expression level of each of the marker genes
B=normalized Ct value of each of the marker genes in respective group
C=normalized Ct value of each of the marker genes in normal control group

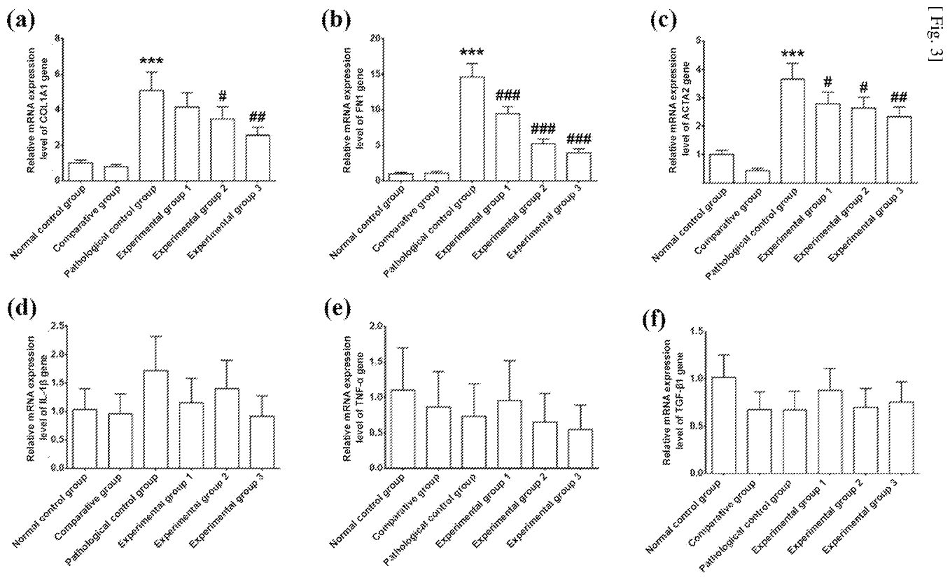

The results are shown in FIG. 3.

FIG. 3 shows the relative mRNA expression levels of six marker genes, i.e., (a) COL1A1, (b) FN1, (c) ACTA2, (d) IL-13, (e) TNF-α, and (f) TGF-β1 in the human bone marrow stromal cells of each group. As shown in FIG. 3 (a) to (c), the relative mRNA expression level of each of COL1A1, FN1, and ACTA2 determined in the pathological control group was significantly higher than that determined in the normal control group, indicating that fibrosis was induced in the human bone marrow stromal cells after incubation with TGF-β1. In addition, the relative mRNA expression levels of COL1A1 determined in the experimental groups 2 and 3 significantly decreased compared with that determined in the pathological control group, and the relative mRNA expression levels of each of FN1 and ACTA2 determined in the experimental groups 1 to 3 significantly decreased compared with that determined in the pathological control group, indicating that treatment with UCMSCs significantly improve fibrosis in the human bone marrow stromal cells. In contrast, as shown in FIG. 3(d) to (f), there were no significant difference in the relative mRNA expression levels of each of IL-1β, TNF-α and TGF-β1 determined in the pathological control group and the experimental groups 1 to 3 compared with that of the normal control group, suggesting that inflammation was not significantly induced in the human bone marrow stromal cells after incubation with TGF-β1 and that UCMSCs did not exert anti-inflammatory effect on the human bone marrow stromal cells.

### B. Effect of UCMSCs on TGF-β1-Induced and MPN Cell-Induced Fibrosis and Inflammation in Human Bone Marrow Stromal Cells

Since MPN mononuclear cells with JAK2^{V617F} mutation have been reported to induce inflammation in human bone marrow stromal cells, in this experiment, human bone marrow stromal cells were incubated with TGF-β1 in combination with HEL 92.1.7 cells, which is a JAK2V^{617F}-positive MPN cell line, so as to mimic post-PV MF cell model, followed by treatment with UCMSCs and determination of the expression levels of gene markers for fibrosis and inflammation.

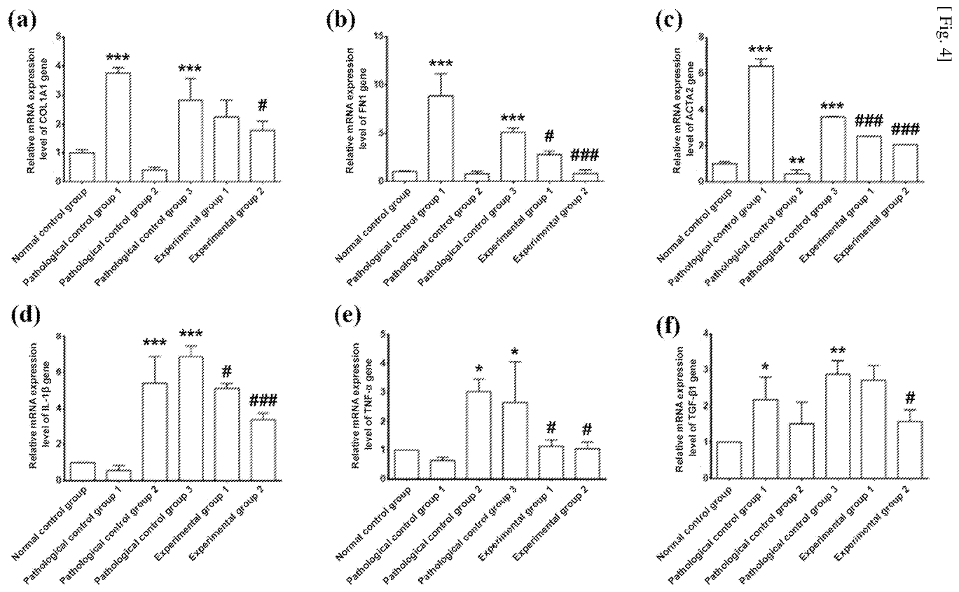

In brief, the human bone marrow stromal cells were divided into 6 groups, namely, a normal control group (NCG), three pathological control groups (PCG1 to PCG3), and 2 experimental groups, i.e., experimental groups 1 and 2 (EG1 and EG2), with the number of cells in each group being 4×10⁴ cells. Next, the human bone marrow stromal cells in the PCG3 and EG1 and EG2 were co-cultivated with 1×10⁵ of HEL 92.1.7 cells in the presence of 5 ng/mL of TGF-β1 and for 72 hours, the human bone marrow stromal cells in the PCG1 were incubated with 5 ng/mL of TGF-β1 for 72 hours, the human bone marrow stromal cells in the PCG2 were co-cultivated with 1×10⁵ of HEL 92.1.7 cells for 72 hours, and the human bone marrow stromal cells in the NCG were not subjected to any treatment. After the HEL 92.1.7 cells were separated from the human bone marrow stromal cells, the human bone marrow stromal cells in the EG1 and EG2 were subjected to treatment with UCMSCs at a ratio of cell number of UCMSCs to human bone marrow stromal cells of 1:4 and 1:2, respectively, for 72 hours, while the human bone marrow stromal cells in the NCG and PCG1 to PCG3 received no treatment. Subsequently, after removal of the UCMSCs, the human bone marrow stromal cells in each group were subjected to total RNA extraction, cDNA synthesis, real-time PCR, normalization of gene expression data, and calculation of the relative mRNA expression levels of COL1A1, FN1, ACTA2, IL-1β, TNF-α, and TGF-β1 using the procedures and conditions as described in section A above. The results are shown in FIG. 4.

FIG. 4 shows the relative expression levels of (a) COL1A1, (b) FN1, (c) ACTA2, (d) IL-1β, (e) TNF-α, and (f) TGF-β1 in the human bone marrow stromal cells of each group. As shown in FIG. 4, the relative expression level of each of COL1A1, FN1, ACTA2, IL-1β, TNF-α, and TGF-β1 determined in the pathological control group 3 was significantly higher than that determined in the normal control group, indicating that fibrosis was induced in the human bone marrow stromal cells after co-cultivation with HEL 92.1.7 cells in the presence of TGF-β1. In addition, the relative mRNA expression level of COL1A1 determined in the experimental group 2 significantly decreased compared with that determined in the pathological control group 3, the relative mRNA expression levels of each of FN1, ACTA2, IL-1β and TNF-α determined in the experimental groups 1 and 2 significantly decreased compared with that determined in the pathological control group 3, and the relative mRNA expression level of TGF-β1 determined in the experimental group 2 significantly decreased compared with that determined in the pathological control group 3, indicating that UCMSCs exert anti-fibrosis and anti-inflammatory effects on the human bone marrow stromal cells.

### C. Effect of UCMSCs on Megakaryocyte-Induced Fibrosis and Inflammation in Human Bone Marrow Stromal Cells

Since megakaryocytes have been reported to induce bone marrow fibrosis and inflammation in post-ET MF and PMF, and since MEG-01 cells has been reported to potentiate fibrosis in human bone marrow stromal cells, in this experiment, human bone marrow stromal cells were co-cultivated with MEG-01 cells, followed by treatment with UCMSCs and determination of the expression levels of gene markers for fibrosis and inflammation.

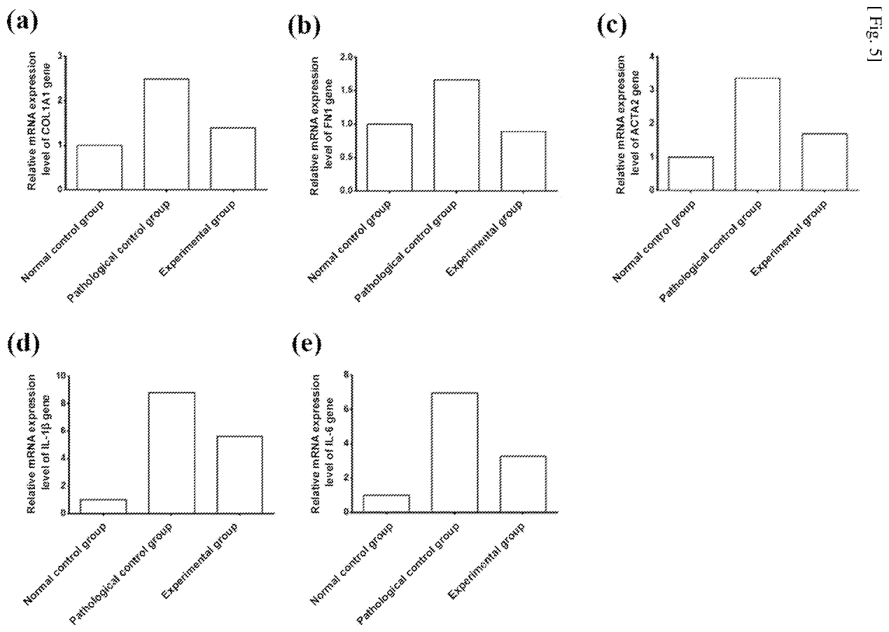

In brief, the human bone marrow stromal cells were divided into 3 groups, namely, a normal control group (NCG), a pathological control group (PCG), and an experimental group (EG), with the number of cells in each group being 4×10⁴ cells. Next, the human bone marrow stromal cells in the PCG and EG were co-cultivated with 1×10⁵ MEG-01 cells for 48 hours, while the human bone marrow stromal cells in the NCG were not subjected to any treatment. After the MEG-01 cells were separated from the human bone marrow stromal cells, the human bone marrow stromal cells in the EG were subjected to treatment with UCMSCs at a ratio of cell number of UCMSCs to human bone marrow stromal cells of 1:1 for 72 hours, while the human bone marrow stromal cells in the NCG and PCG received no treatment. Subsequently, after removal of the UCMSCs, the human bone marrow stromal cells in each group were subjected to total RNA extraction, cDNA synthesis, real-time PCR, normalization of gene expression data, and calculation of the relative mRNA expression levels of COL1A1, FN1, ACTA2, IL-1β, and IL-6 using the procedures and conditions as described in section A above. The results are shown in FIG. 5.

FIG. 5 shows the relative expression levels of (a) COL1A1, (b) FN1, (c) ACTA2, (d) IL-1β, and (e) IL-6 in the human bone marrow stromal cells of each group. As shown in FIG. 5, the relative expression level of each of COL1A1, FN1, ACTA2, IL-1β, and IL-6 determined in the pathological control group was higher than that determined in the normal control group, indicating that fibrosis and inflammation were induced in the human bone marrow stromal cells after co-cultivation with MEG-01 cells. In addition, the relative mRNA expression level of each of COL1A1, FN1, ACTA2, IL-1β, and IL-6 determined in the experimental group decreased compared with that determined in the pathological control group, indicating that UCMSCs exert anti-fibrosis and anti-inflammatory effects on the human bone marrow stromal cells.

Since IL-1β was reported to mediate myelofibrosis in SET-2 cells, which are megakaryocytes with JAK2V^{617F} mutation, human bone marrow stromal cells were co-cultivated with SET-2 cells in the presence of IL-1β, followed by treatment with UCMSCs and determination of the expression levels of gene markers for fibrosis and inflammation.

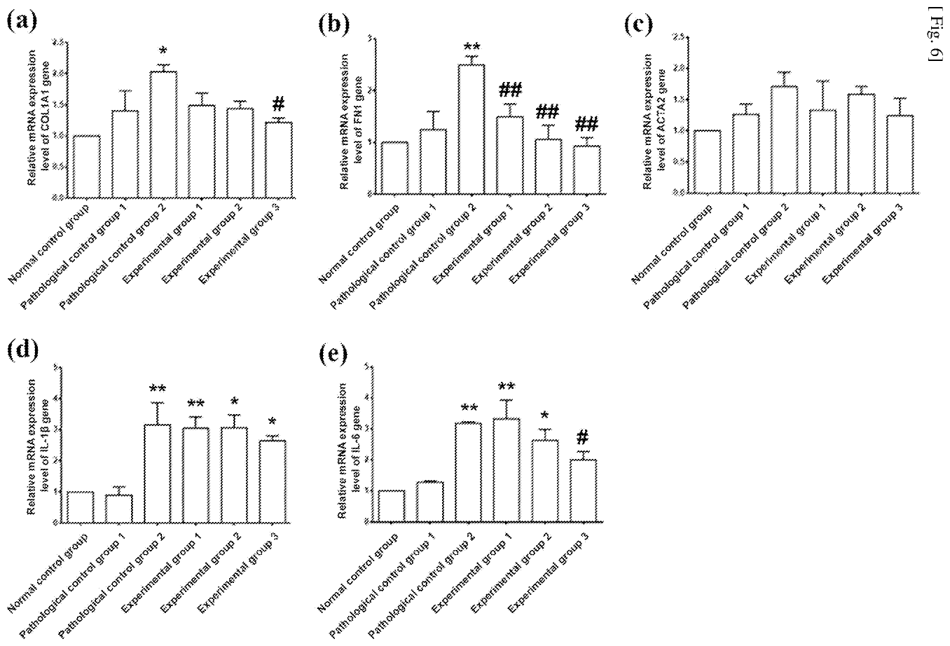

In brief, the human bone marrow stromal cells were divided into 6 groups, namely, a normal control group (NCG), two pathological control groups (PCG1 and PCG2), and 3 experimental groups, i.e., experimental groups 1 to 3 (EG1 to EG3), with the number of cells in each group being 4×10⁴ cells. Next, the human bone marrow stromal cells in a respective one of the PCG2 and EG1 to EG3 were co-cultivated with 1×10⁵ of IL-1β-pretreated SET-2 cells (note: the SET-2 cells were first treated with 30 ng/mL of IL-1β for 24 hours and then the IL-1β was removed therefrom) for 72 hours, the human bone marrow stromal cells in the PCG1 were co-cultivated with 1×10⁵ of SET-2 cells for 72 hours, and the human bone marrow stromal cells in the NCG were not subjected to any treatment. After the SET-2 cells were separated from the human bone marrow stromal cells, the human bone marrow stromal cells in the EG1 to EG3 were subjected to treatment with UCMSCs at a ratio of cell number of UCMSCs to human bone marrow stromal cells of 1:10, 1:4 and 1:2, respectively, for 72 hours, while the human bone marrow stromal cells in the NCG, PCG1 and PCG2 received no treatment. Subsequently, after removal of the UCMSCs, the human bone marrow stromal cells in each group were subjected to total RNA extraction, cDNA synthesis, real-time PCR, normalization of gene expression data, and calculation of the relative mRNA expression levels of COL1A1, FN1, ACTA2, IL-1, and IL-6 using the procedures and conditions as described in section A above. The results are shown in FIG. 6.

FIG. 6 shows the relative expression levels of (a) COL1A1, (b) FN1, (c) ACTA2, (d) IL-13, and (e) IL-6 in the human bone marrow stromal cells of each group. As shown in FIG. 6, the relative expression level of each of COL1A1, FN1, IL-1β and IL-6 determined in the pathological control group 2 was significantly higher than that determined in the normal control group, indicating that fibrosis and inflammation were induced in the human bone marrow stromal cells after co-cultivation with IL-1β-pretreated SET-2 cells. In addition, the relative mRNA expression level of COL1A1 determined in the experimental group 3 significantly decreased compared with that determined in the pathological control group, the relative mRNA expression level of FN1 determined in the experimental groups 1 to 3 significantly decreased compared with that determined in the pathological control group, and the relative mRNA expression level of IL-6 determined in the experimental group 3 significantly decreased compared with that determined in the pathological control group, indicating that UCMSCs exert anti-fibrosis and anti-inflammatory effects on the human bone marrow stromal cells.

### D. Effect of UCMSCs on Human Bone Marrow Stromal Cells-Induced Inflammation in MPN Cells

Since human bone marrow stromal cells were reported to induce release of inflammatory cytokines from megakaryocytes in post-ET MF and PMF, which is important in the pathogenic progression of myelofibrosis, in this experiment, an MPN cell line, i.e., MEG-01 cells, were incubated with human bone marrow stromal cells, followed by treatment with UCMSCs and determination of the expression levels of gene markers for inflammation.

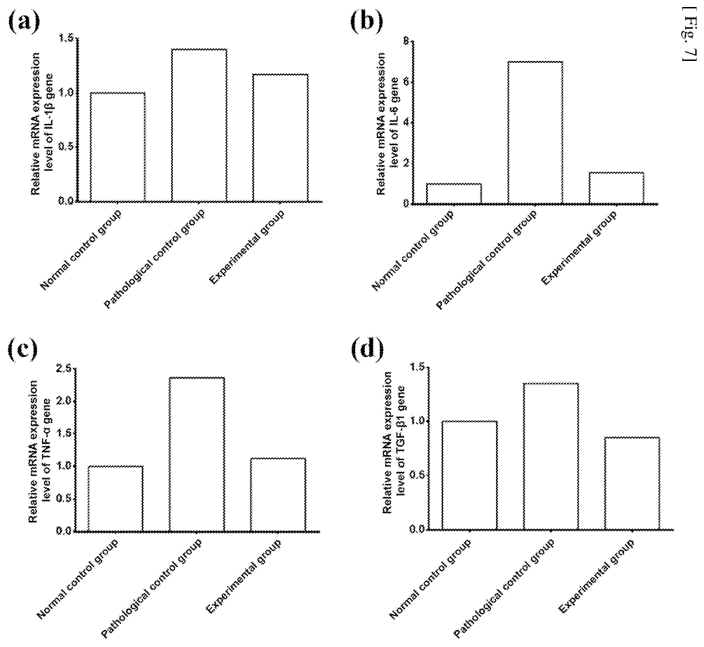

In brief, the MEG-01 cells were divided into 3 groups, namely, a normal control group (NCG), a pathological control group (PCG), and an experimental group (EG), with the number of MEG-01 cells in each group being 1×10⁵ cells. Next, the MEG-01 cells in the PCG and EG were co-cultivated with 4×10⁴ of human bone marrow stromal cells for 48 hours to induce inflammatory response, while the MEG-01 cells in the NCG were not subjected to any treatment. After the human bone marrow stromal cells were separated from the MEG-01, the MEG-01 in the EG were subjected to treatment with 4×10⁴ of UCMSCs at a ratio of cell number of UCMSCs to MEG-01 of 1:1 for 72 hours, while the MEG-01 cells in the NCG and PCG received no treatment. Subsequently, after removal of the UCMSCs, the MEG-01 cells in each group were subjected to total RNA extraction, cDNA synthesis, real-time PCR, normalization of gene expression data, and calculation of the relative mRNA expression levels of IL-1β, IL-6, TNF-α and TGF-β1 using the procedures and conditions as described in section A above. The results are shown in FIG. 7.

FIG. 7 shows the relative expression levels of (a) IL-1β, (b) IL-6, (c) TNF-α and (d) TGF-β1 in the MEG-01 cells of each group. As shown in FIG. 7, the relative expression level of each of IL-1β, IL-6, TNF-α and TGF-β1 determined in the pathological control group was higher than that determined in the normal control group, indicating that inflammation was induced in the MEG-01 cells after co-cultivation with the human bone marrow stromal cells. In addition, the relative mRNA expression level of each of IL-1β, IL-6, TNF-α and TGF-β1 determined in the experimental group decreased compared with that determined in the pathological control group, indicating that UCMSCs exert anti-inflammatory effect on the MEG-01 cells.

### E. Effect of UCMSCs on Cytokine-Induced Inflammation in MPN Cells

Since IL-1β has been reported to mediate myelofibrosis in post-PV cell model, in this experiment, another MPN cell line, i.e., HEL 92.1.7 cells were incubated with IL-1β, followed by treatment with UCMSCs and determination of the expression levels of gene markers for inflammation.

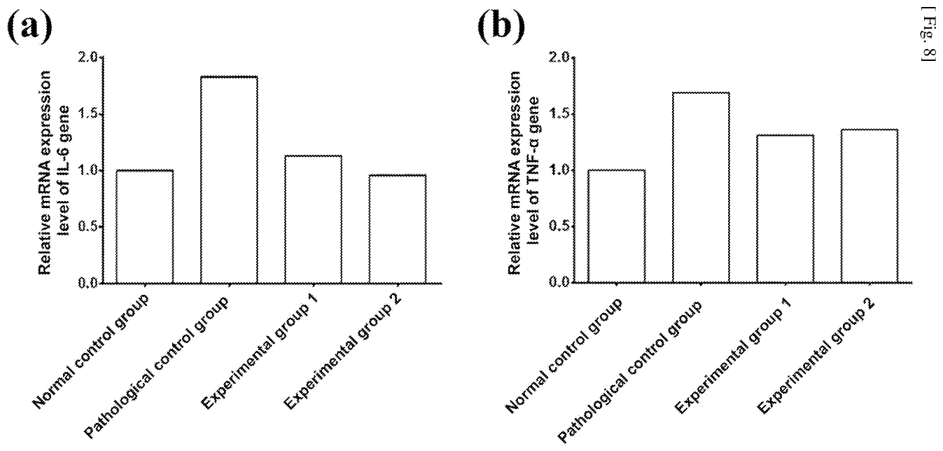

In brief, the HEL 92.1.7 cells were divided into 4 groups, namely, a normal control group (NCG), a pathological control group (PCG), and two experimental groups, i.e., experimental groups 1 and 2 (EG1 and EG2), with the number of HEL 92.1.7 cells in each group being 2.5×10⁴ cells. Next, the HEL 92.1.7 cells in the PCG, EG1 and EG2 were incubated with 30 ng/mL of IL-1β for 72 hours to induce inflammatory response, while the HEL 92.1.7 cells in the NCG were not subjected to any treatment. Afterwards, the HEL 92.1.7 cells in the EG1 and EG2 were subjected to treatment with UCMSCs at a ratio of cell number of UCMSCs to HEL 92.1.7 of 1:4 and 1:2, respectively, for 48 hours, while the HEL 92.1.7 cells in the NCG received no treatment. Subsequently, after removal of the UCMSCs, the HEL 92.1.7 cells in each group were subjected to total RNA extraction, cDNA synthesis, real-time PCR, normalization of gene expression data, and calculation of the relative mRNA expression levels of IL-6 and TNF-α using the procedures and conditions as described in section A above. The results are shown in FIG. 8.

FIG. 8 shows the relative expression levels of (a) IL-6 and (b) TNF-α in the HEL 92.1.7 cells of each group. As shown in FIG. 8, the relative expression level of each of IL-6 and TNF-α determined in the pathological control group was higher than that determined in the normal control group, indicating that inflammation was induced in the HEL 92.1.7 cells after incubation with IL-1β. In addition, the relative mRNA expression level of each of IL-6 and TNF-α determined in the experimental groups 1 and 2 decreased compared with that determined in the pathological control group, indicating UCMSCs exert anti-inflammatory effect on the HEL 92.1.7 cells.

### F. Effect of Different Sources-Derived MSCs on TGF-β1-Induced Fibrosis in Human Bone Marrow Stromal Cells

In order to validate the anti-fibrosis effect of MSCs, human bone marrow stromal cells with fibrosis induced by TGF-β1 were treated with different sources-derived MSCs, including umbilical cord-derived MSCs (UCMSCs), adipose tissue-derived MSCs (AMSCs), and lung-derived MSCs (LMSCs), followed by determination of the expression levels of gene markers for fibrosis.

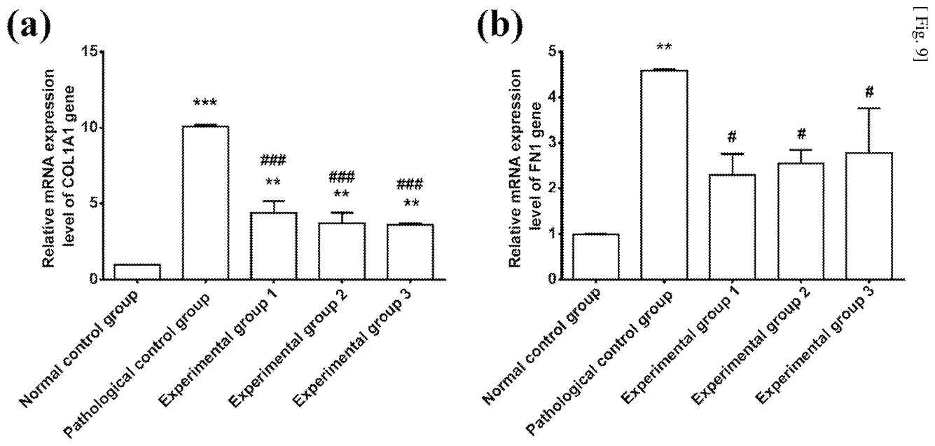

In brief, the human bone marrow stromal cells were divided into 5 groups, namely, a normal control group (NCG), a pathological control group (PCG), and 3 experimental groups, i.e., experimental groups 1 to 3 (EG1 to EG3), with the number of human bone marrow stromal cells in each group being 4×10⁴ cells. Next, the human bone marrow stromal cells in the PCG and EG1 to EG3 were incubated with 5 ng/mL of TGF-β1 for 72 hours, while the human bone marrow stromal cells in the NCG were not subjected to any treatment. Afterwards, the human bone marrow stromal cells in the EG1 to EG3 were respectively subjected to treatment with 2×10⁴ of UCMSCs, 2×10⁴ of AMSCs and 2×10⁴ of LMSCs for 72 hours, while the human bone marrow stromal cells in the NCG and PCG received no treatment. Subsequently, after removal of the UCMSCs, the human bone marrow stromal cells in each group were subjected to total RNA extraction, cDNA synthesis, real-time PCR, normalization of gene expression data, and calculation of the relative mRNA expression levels of COL1A1 and FN1 using the procedures and conditions as described in section A above. The results are shown in FIG. 9.

FIG. 9 shows the relative expression levels of (a) COL1A1 and (b) FN1 in the human bone marrow stromal cells of each group. As shown in FIG. 9, the relative expression level of each of COL1A1 and FN1 determined in the pathological control group was significantly higher than that determined in the normal control group, indicating that fibrosis was induced in the human bone marrow stromal cells after incubation with TGF-β1. In addition, the relative mRNA expression level of each of COL1A1 and FN1 determined in the experimental groups 1 to 3 decreased compared with that determined in the pathological control group, indicating that a respective one UCMSCs, AMSCs and LMSCs are capable of exerting anti-fibrosis effect on the human bone marrow stromal cells.

### G. Effect of Different Sources-Derived MSCs on MPN Cell-Induced Fibrosis in Human Bone Marrow Stromal Cells

In order to further validate the anti-fibrosis effect of MSCs, human bone marrow stromal cells with fibrosis induced MPN cells in the presence of IL-1β were subjected to treatment with different sources-derived MSCs, including UCMSCs, AMSCs, and LMSCs, followed by determination of the expression levels of gene markers for fibrosis.

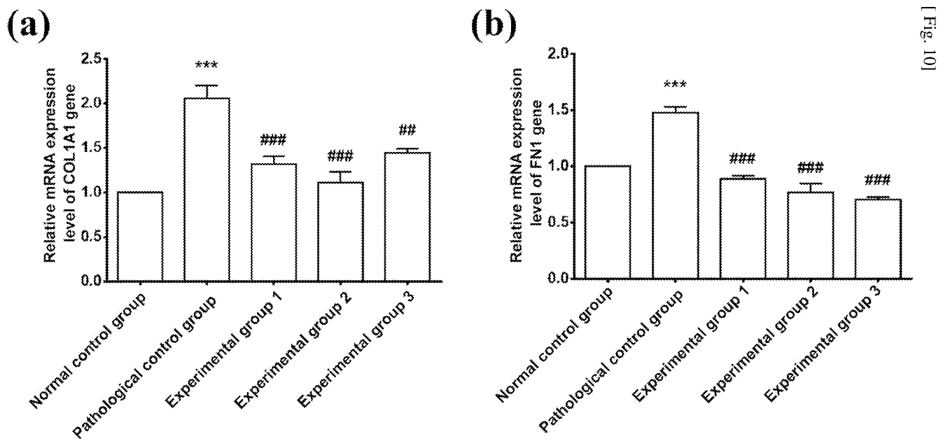

In brief, the human bone marrow stromal cells were divided into 5 groups, namely, a normal control group (NCG), a pathological control group (PCG), and 3 experimental groups, i.e., experimental groups 1 to 3 (EG1 to EG3), with the number of the human bone marrow stromal cells in each group being 4×10⁴ cells. Next, the human bone marrow stromal cells in a respective one of the PCG and EG1 to EG3 were co-cultivated with 1×10⁵ of IL-1β-pretreated SET-2 cells (note: the SET-2 cells were first treated with 30 ng/mL of IL-1β for 24 hours and then the IL-1β was removed therefrom) for 72 hours, while the human bone marrow stromal cells in the NCG were not subjected to any treatment. After the SET-2 cells were separated from the human bone marrow stromal cells, the human bone marrow stromal cells in the EG1 to EG3 were respectively subjected to treatment with 2×10⁴ of UCMSCs, 2×10⁴ of AMSCs and 2×10⁴ of LMSCs for 72 hours, while the human bone marrow stromal cells in the NCG and PCG received no treatment. Subsequently, after removal of the respective one of the UCMSCs, AMSCs and LMSCs, the human bone marrow stromal cells in each group were subjected to total RNA extraction, cDNA synthesis, real-time PCR, normalization of gene expression data, and calculation of the relative mRNA expression levels of COL1A1 and FN1 using the procedures and conditions as described in section A above. The results are shown in FIG. 10.

FIG. 10 shows the relative expression levels of (a) COL1A1 and (b) FN1 in the human bone marrow stromal cells of each group. As shown in FIG. 10, the relative expression level of each of COL1A1 and FN1 determined in the pathological control group was significantly higher than that determined in the normal control group, indicating that fibrosis were induced in the human bone marrow stromal cells after co-cultivation with IL-1β-pretreated SET-2 cells. In addition, the relative mRNA expression level of each of COL1A1 and FN1 determined in the experimental groups 1 to 3 decreased compared with that determined in the pathological control group, indicating that a respective one UCMSCs, AMSCs and LMSCs are capable of exerting anti-fibrosis effect on the human bone marrow stromal cells.

### Example 3. Evaluation of Effect of Primed UCMSCs on Alleviation of Myelofibrosis

In this example, primed UCMSCs, i.e., indoleamine-pyrrole 2,3-dioxygenase-positive (IDO)-expressing UCMSCs, were prepared and then subjected to evaluation using the cellular model of bone marrow fibrosis as described in Example 2 above, so as to determine the efficacy of the primed UCMSCs against fibrosis.

### A. Preparation of Primed UCMSCs

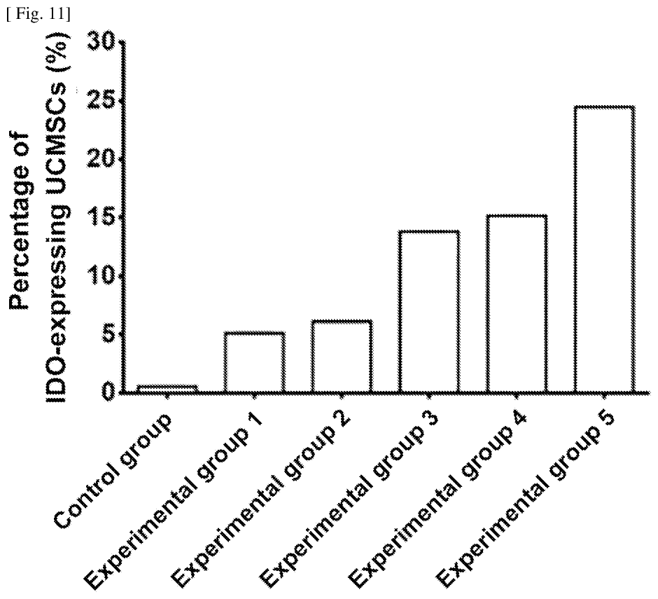

UCMSCs culture as described in Item 2 of the General Experimental Materials were divided into 6 groups, namely, a control group (CG), and 5 experimental groups, i.e., experimental group 1 to 5 (EG1 to EG5), with the number of UCMSCs in each group being 5.8×10⁵ cells. Next, the UCMSCs in the EG1 to EG5 were subjected to priming by treating with different concentrations of IFN-γ, i.e., 50, 100, 200, 250 and 500 U/mL of IFN-γ, respectively, for 72 hours, while the UCMSCs in the CG were not subjected to any treatment. For identification of IDO-expressing UCMSCs, the cells in each group were stained with phycoerythrin-conjugated IDO monoclonal antibody (Manufacturer: Thermo Fisher Scientific; Catalogue no.: 12-9477-42) while phycoerythrin-conjugated mouse IgG1 kappa isotype control (Manufacturer: Thermo Fisher Scientific; Catalogue no.: 12-4714-82) served as the control antibody, followed by subjecting the stained cells to flow cytometry conducted using CytoFLEX Flow Cytometer (Beckman Coulter), so as to determine the percentage of IDO-expressing UCMSCs in each group. The results are shown in FIG. 11.

FIG. 11 shows the percentage of IDO-expressing UCMSCs in each group after treatment with different concentrations of IFN-γ for 72 hours. As shown in FIG. 11, the percentage of IDO-expressing UCMSCs, i.e., primed UCMSCs, determined in each of experimental groups 1 to 5 was higher compared with that of the control group, and such increase was dependent on the concentration of IFN-γ.

Since the aforesaid results showed that UCMSCs treated with 250 and 500 U/mL of IFN-γ had higher percentage of IDO-expressing UCMSCs, in the following experiment, UCMSCs were treated with these concentrations of IFN-γ at different time periods to determine the percentage of IDO-expressing UCMSCs.

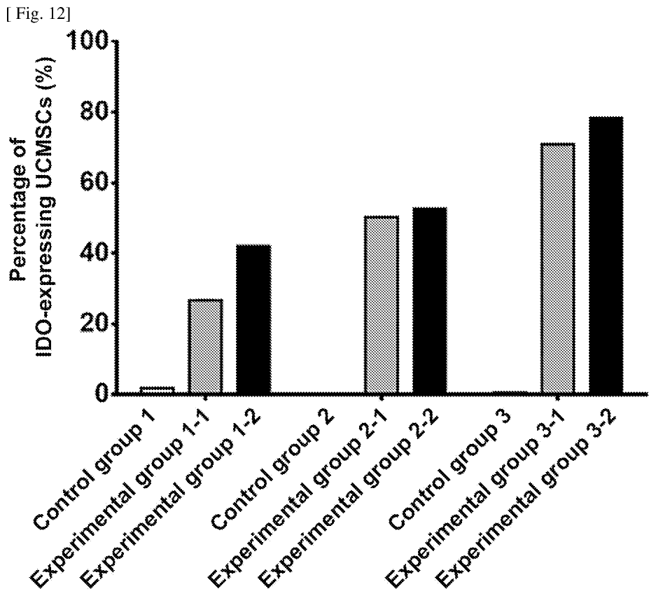

In brief, the UCMSCs culture as described in Item 2 of the General Experimental Materials were divided into 9 groups, namely, three control groups, i.e., control groups 1 to 3 (CG1 to CG3), and 6 experimental groups, i.e., experimental group 1-1 (EG1-1), experimental group 1-2 (EG1-2), experimental group 2-1 (EG2-1), experimental group 2-2 (EG2-2), experimental group 3-1 (EG3-1) and experimental group 3-2 (EG3-2), with the number of UCMSCs in each group being 5.8×10⁵ cells. Next, the UCMSCs in the EG1-1, EG2-1, and EG3-1 were subjected to priming by treating with 250 U/mL of IFN-γ for 24 hours, 48 hours, and 72 hours, respectively, the UCMSCs in EG1-2, EG2-2, and EG3-2 were treated with 500 U/mL of IFN-γ for 24 hours, 48 hours, and 72 hours, respectively, while the UCMSCs in the CG1, CG2 and CG3 were not subjected to any treatment and were left to stand for 24 hours, 48 hours, and 72 hours, respectively. Thereafter, the thus obtained primed UCMSCs in each group were subjected to flow cytometry conducted according to the aforesaid procedures so as to determine the percentage of IDO-expressing UCMSCs in each group. The results are shown in FIG. 12.

FIG. 12 shows the percentage of IDO-expressing UCMSCs in each group after treatment with IFN-γ at different concentrations for different time periods. As shown in FIG. 12, the percentages of IDO-expressing UCMSCs, i.e., primed UCMSCs, determined in the experimental groups 1-1 and 1-2 were higher compared with that of the control group 1, the percentages of IDO-expressing UCMSCs determined in the experimental groups 2-1 and 2-2 were higher compared with that of the control group 2, and the percentages of IDO-expressing UCMSCs determined in the experimental groups 3-1 and 3-2 were higher compared with that of the control group 3. In addition, the percentage of IDO-expressing UCMSC determined in the experimental group 3-1 was higher compared with those of the experimental groups 2-1 and 1-1, while the percentage of percentage of IDO-expressing UCMSCs determined in the experimental group 3-2 was higher compared with those of the experimental groups 2-2 and 1-2. These results suggest that the increase in the percentage of IDO-expressing UCMSCs was dependent on the concentration of IFN-γ and time period of treatment.

In order to determine whether addition of a supplement, which is selected from one of vitamin, steroid, cytokine, double-stranded RNA, and histone deacetylase (HDAC) inhibitor, enhanced the effect of IFN-γ in the priming of UCMSCs, the following experiments were conducted.

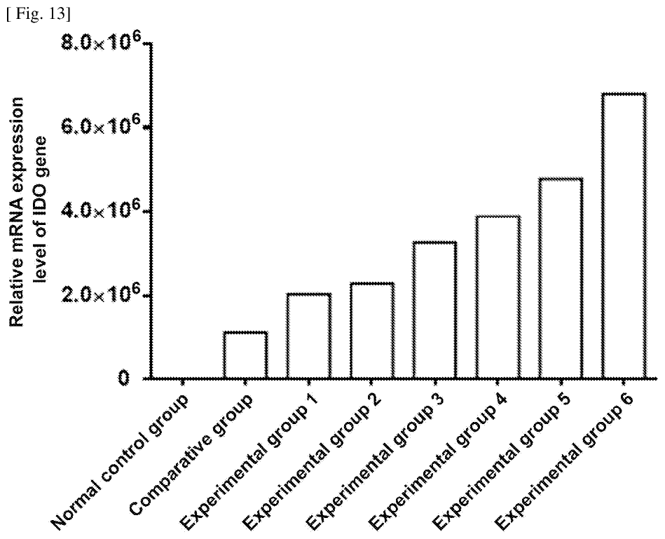

In brief, UCMSCs culture as described in Item 2 of the General Experimental Materials were divided into 8 groups, namely, a normal control group (NCG), a comparative group (CG) and 6 experimental groups, i.e., experimental group 1 to experimental group 6 (EG1 to EG6), with the number of UCMSCs in each group being 5.8×10⁵ cells. Next, the UCMSCs in each group were subjected to priming, in which the UCMSCs in the CG were treated with 250 U/mL of IFN-γ for 72 hours, the UCMSCs in a respective one of the EG1 to EG6 were treated with 250 U/mL of IFN-γ in combination with 1 μM of retinoic acid, 1 μg/mL of dexamethasone, 10 ng/mL of TNF-α, 25 μg/mL of polyinosinic acid-polycytidylic acid, 0.5 mM of valproic acid, and 1 μM of budesonide, respectively, for 72 hours, while the UCMSCs in the NCG were not subjected to any treatment and were left to stand for 72 hours. Thereafter, the thus obtained primed UCMSCs in each group were subjected to total RNA extraction, cDNA synthesis, real-time PCR using the primer pair specific for IDO gene as shown in Table 3 below, normalization of gene expression data, and calculation of the relative mRNA expression level of IDO gene using the procedures and conditions as described in section A of Example 2. The results are shown in FIG. 13.

TABLE 3

| | | | Size of |
|---|---|---|---|
| | | | PCR |
| | | Nucleotide sequence | product |
| Marker gene | PCR primer | (5′->3′) | (bp) |
| IDO gene | Forward primer | gcaagaacgggacactttgc | 167 |
| (GenBank accession | IDO-F | (SEQ ID NO. 17) | |
| no. NM_002164) | Reverse primer | cgtctgatagotgggggttg | |
| | IDO-R | (SEQ ID NO. 18) | |

FIG. 13 shows the relative mRNA expression level of IDO gene in each group after treatment with IFN-γ in combination with a respective one of the supplement. As shown in FIG. 13, the relative mRNA expression level of IDO gene determined in each of the experimental groups 1 to 6 was higher than that determined in the comparative group, indicating that the number of primed UCMSCs determined after the UCMSCs were treated with IFN-γ in combination with a respective one of retinoic acid, dexamethasone, TNF-α, polyinosinic acid-polycytidylic acid, valproic acid, and budesonide was even greater compared with the number of primed UCMSCs determined after the UCMSCs were treated with IFN-γ only. These results suggest that the respective one of retinoic acid, dexamethasone, TNF-α, polyinosinic acid-polycytidylic acid, valproic acid, and budesonide is capable of enhancing the effect of IFN-γ in the priming of UCMSCs.

Since the aforesaid result demonstrate that UCMSCs treated with IFN-γ in combination with budesonide showed the greatest increase in the relative mRNA expression level of IDO gene, the following experiment was conducted to determine the effect of treatment of UCMSCs with IFN-γ and budesonide for different time periods on the percentage of IDO-expressing UCMSCs.

In brief, UCMSCs culture as described in Item 2 of the General Experimental Materials were divided into 8 groups, namely, three control groups, i.e., control groups 1 to 3 (CG1 to CG3), and 5 experimental groups, i.e., experimental group 1-1 (EG1-1), experimental group 1-2 (EG1-2), experimental group 2-1 (EG2-1), experimental group 2-2 (EG2-2) and experimental group 3-2 (EG3-2), with the number of UCMSCs in each group being 5.8×10⁵ cells. The priming process was conducted by treating the UCMSCs with 250 U/mL of IFN-γ and/or 1 μM of budesonide for a total time period of 72 hours. The time period of treatment with IFN-γ and/or budesonide for the UCMSCs in each group are shown in Table 4 below.

TABLE 4

| Group | IFN-γ (250 U/mL) | Budesonide (1 μM) |
|---|---|---|
| CG1 | 24 hours | — |
| CG2 | 48 hours | — |
| CG3 | 72 hours | — |
| EG1-1 | 24 hours | 48 hours |
| EG1-2 | 24 hours | 72 hours |
| EG2-1 | 48 hours | 48 hours |
| EG2-2 | 48 hours | 72 hours |
| EG3-2 | 72 hours | 72 hours |

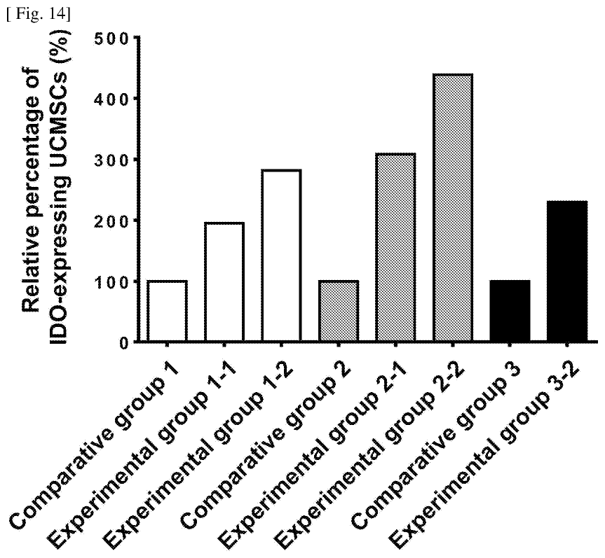

Thereafter, the thus obtained primed UCMSCs in each group were subjected to flow cytometry conducted according to the aforesaid procedures so as to determine the relative percentage of IDO-expressing UCMSCs in each group. The results are shown in FIG. 14.

FIG. 14 shows the relative percentage of IDO-expressing UCMSCs in each group after treatment with IFN-γ for different time period in combination with budesonide for different time period. As shown in FIG. 14, the relative percentages of IDO-expressing UCMSCs, i.e., primed UCMSCs, determined in the experimental groups 1-1, 1-2, 2-1, 2-2 and 3-2 were higher compared with those determined in the comparative groups 1 to 3, indicating that budesonide is capable of enhancing the effect of IFN-γ in the priming of UCMSCs. In addition, the percentage of IDO-expressing UCMSCs determined in the experimental group 1-2 was higher compared with that determined in the experimental group 1-1, and the percentage of IDO-expressing UCMSCs determined in the experimental group 2-2 was higher compared with that determined in the experimental group 2-1, demonstrating that treatment with budesonide for a relatively longer time period further enhances the effect of IFN-γ in the priming of UCMSCs.

### B. Effect of Primed UCMSCs on TGF-β1-Induced and MPN Cell-Induced Fibrosis in Human Bone Marrow Stromal Cells

In order to determine the effect of primed UCMSCs versus the effect of normal (i.e. non-primed) UCMSCs in post-PV MF cell model, in this experiment, human bone marrow stromal cells were co-cultivated with HEL 92.1.7 cells in the presence of TGF-β1, followed by treatment with normal UCMSCs or primed UCMSCs and determination of the expression levels of gene markers for fibrosis in the human bone marrow stromal cells.

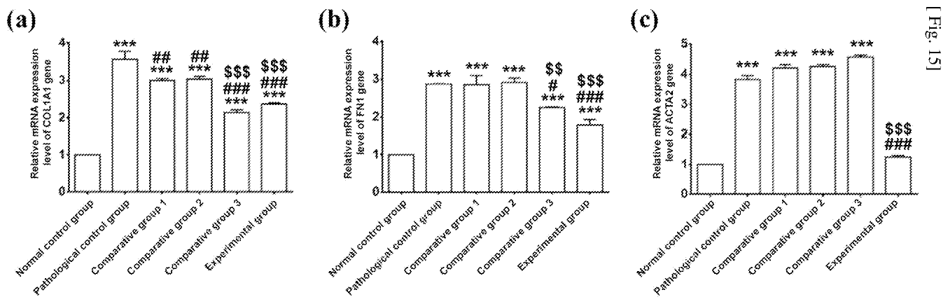

In brief, human bone marrow stromal cells were divided into 6 groups, namely, a normal control group (NCG), a pathological control groups (PCG), three comparative groups, i.e., comparative groups 1 to 3 (CG1 to CG3) and an experimental groups (EG), with the number of human bone marrow stromal cells in each group being 4×10⁴ cells. Next, the human bone marrow stromal cells in the PCG, CG1, CG2, CG3 and EG were subjected to induction of fibrosis by co-cultivation with 4×10⁴ HEL 92.1.7 cells in the presence of 5 ng/mL of TGF-β1 for 72 hours, while the human bone marrow stromal cells in the NCG were not subjected to any treatment. After the HEL 92.1.7 cells were separated from the human bone marrow stromal cells, the human bone marrow stromal cells in the CG1 to CG3 were treated with normal UCMSCs at a ratio of cell number of normal UCMSCs to human bone marrow stromal cells of 1:10, 1:4 and 1:2, respectively, for 72 hours, the human bone marrow stromal cells in the EG was treated with primed UCMSCs at a ratio of cell number of primed UCMSCs to human bone marrow stromal cells of 1:10 for 72 hours, while human bone marrow stromal cells in the NCG and PCG received no treatment. Subsequently, after removal of the normal UCMSCs or the primed UCMSCs, the human bone marrow stromal cells in each group were subjected to total RNA extraction, cDNA synthesis, real-time PCR, normalization of gene expression data, and calculation of the relative mRNA expression levels of COL1A1, FN1, and ACTA2 using the procedures and conditions as described in section A of Example 2 above. The results are shown in FIG. 15.

FIG. 15 shows the relative expression levels of (a) COL1A1, (b) FN1 and (c) ACTA2 in the human bone marrow stromal cells of each group. As shown in FIG. 15, the relative expression level of each of COL1A1, FN1, and ACTA2 determined in the pathological control group was significantly higher than that determined in the normal control group, indicating that fibrosis was induced in the human bone marrow stromal cells after co-cultivation with HEL 92.1.7 cells in the presence of TGF-β1. In addition, the relative mRNA expression level of COL1A1 determined in the comparative groups 1 to 3 significantly decreased compared with that determined in the pathological control group, and the relative mRNA expression level of FN1 determined in the comparative group 3 significantly decreased compared with that determined in the pathological control group, indicating that treatment with normal UCMSCs significantly improve fibrosis in the human bone marrow stromal cells. Moreover, the relative mRNA expression level of each of COL1A1, FN1 and ACTA2 determined in the experimental group significantly decreased compared with that determined in the comparative group 1, indicating that in comparison with normal UCMSCs, primed UCMSCs demonstrated an enhanced efficacy against fibrosis in human bone marrow stromal cells.

### C. Effect of Primed UCMSCs on Megakaryocytes-Induced Fibrosis in Human Bone Marrow Stromal Cells

In order to determine the effect of primed UCMSCs versus the effect of normal (i.e. non-primed) UCMSCs in post-ET MF and PMF cell model, in this experiment, human bone marrow stromal cells were co-cultivated with SET-2 cells in the presence of IL-1β, followed by treatment with normal UCMSCs or primed UCMSCs and determination of the expression levels of gene markers for fibrosis.

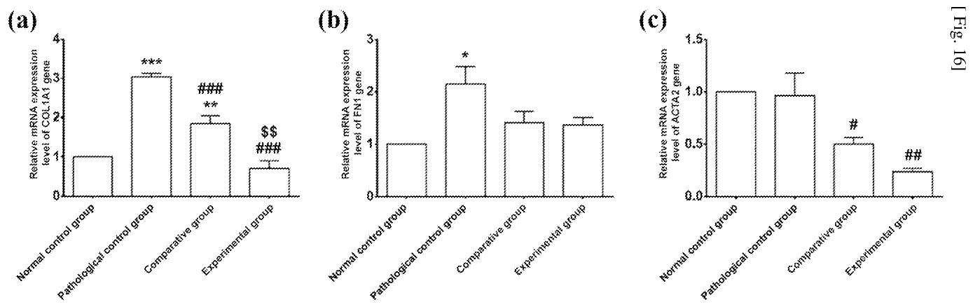

In brief, the human bone marrow stromal cells were divided into 4 groups, namely, a normal control group (NCG), a pathological control group (PCG), a comparative group (CG), and an experimental group (EG), with the number of human bone marrow stromal cells in each group being 4×10⁴ cells. Next, the human bone marrow stromal cells in a respective one of the PCG, CG and EG were co-cultivated with 1×10⁵ of IL-1β-pretreated SET-2 cells (note: the SET-2 cells were first treated with 30 ng/mL of IL-1β for 24 hours and then the IL-1β was removed therefrom) for 72 hours, while the human bone marrow stromal cells in the NCG were not subjected to any treatment. After the SET-2 cells were separated from the human bone marrow stromal cells, the human bone marrow stromal cells in the CG were treated with normal UCMSC at a ratio of cell number of normal UCMSCs to human bone marrow stromal cells of 1:2 for 72 hours, the human bone marrow stromal cells in the EG was treated with primed UCMSCs at a ratio of cell number of primed UCMSCs to human bone marrow stromal cells of 1:2 for 72 hours, while human bone marrow stromal cells in the NCG and PCG received no treatment. Subsequently, after removal of the normal UCMSCs or the primed UCMSCs, the human bone marrow stromal cells in each group were subjected to total RNA extraction, cDNA synthesis, real-time PCR, normalization of gene expression data, and calculation of the relative mRNA expression levels of COL1A1, FN1, and ACTA2 using the procedures and conditions as described in section A of Example 2 above. The results are shown in FIG. 16.

FIG. 16 shows the relative expression levels of (a) COL1A1, (b) FN1 and (c) ACTA2 in the human bone marrow stromal cells of each group. As shown in FIG. 16, the relative expression level of each of COL1A1 and FN1 determined in the pathological control group was significantly higher than that determined in the normal control group, indicating that fibrosis was induced in the human bone marrow stromal cells after co-cultivation with IL-1β-pretreated SET-2 cells. In addition, the relative mRNA expression level of each of COL1A1 and ATCA2 determined in the comparative group significantly decreased compared with that determined in the pathological control group, indicating that treatment with normal UCMSCs significantly improve fibrosis in the human bone marrow stromal cells. Moreover, the relative mRNA expression level of COL1A1 determined in the experimental group significantly decreased compared with that determined in the comparative group, indicating that in comparison with normal UCMSCs, primed UCMSCs demonstrated an enhanced efficacy against fibrosis in human bone marrow stromal cells.

In summary, the aforesaid results demonstrated that MSCs are capable of alleviating the three major clinical symptoms of myelofibrosis, i.e., anemia, splenomegaly and bone marrow fibrosis, when administered to a myelofibrosis mouse model. In addition, MSCs exhibited anti-fibrosis and anti-inflammatory effects in cellular models of bone marrow fibrosis, as shown by reduction of relative mRNA expression levels of gene markers for fibrosis and inflammation. Furthermore, in comparison to normal MSCs (i.e., non-primed MSCs), primed MSCs demonstrated greater efficacy against fibrosis in such cellular models of bone marrow fibrosis. Therefore, MSCs are expected to have a high potential to be used as a therapeutic agent for alleviating bone marrow fibrosis in myelofibrosis.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

1-13. (canceled)

14. A method for alleviating myelofibrosis, comprising administering to a subject in need thereof a composition including mesenchymal stem cells (MSCs).

15. The method as claimed in claim 14, wherein the MSCs are selected from the group consisting of umbilical cord-derived mesenchymal stem cells (UCMSCs), bone marrow-derived mesenchymal stem cells (BMMSCs), adipose tissue-derived mesenchymal stem cells (AMSCs), dermis-derived mesenchymal stem cells (DMSCs), epidermis-derived mesenchymal stem cells (EMSCs), synovial membrane-derived mesenchymal stem cells (SMMSCs), dental tissue-derived mesenchymal stem cells (dental MSCs), lung-derived mesenchymal stem cells (LMSCs), and combinations thereof.

16. The method as claimed in claim 15, wherein the MSCs are UCMSCs.

17. The method as claimed in claim 16, wherein the UCSMCs are non-primed UCMSCs.

18. The method as claimed in claim 16, wherein the UCSMCs are primed UCMSCs.

19. The method as claimed in claim 18, wherein the primed UCSMCs are indoleamine-pyrrole 2,3-dioxygenase (IDO)-expressing UCMSCs.

20. The method as claimed in claim 19, wherein the IDO-expressing UCMSCs are prepared by cultivation of non-primed UCMSCs in a culture medium supplemented with IFN-γ.

21. The method as claimed in claim 20, wherein the culture medium is further supplemented with a substance selected from the group consisting of vitamin, steroid, cytokine, double-stranded RNA, and histone deacetylase (HDAC) inhibitor.

22. The method as claimed in claim 21, wherein the vitamin is retinoic acid, the steroid is dexamethasone or budesonide, the cytokine is TNF-α, the double-stranded RNA is polyinosinic acid-polycytidylic acid, and the HDAC inhibitor is valproic acid.

23. The method as claimed in claim 14, wherein the subject is not subjected to myeloablative transplantation.

24. The method as claimed in claim 14, wherein the myelofibrosis is selected from the group consisting of primary myelofibrosis, post-essential thrombocythemia myelofibrosis, post-polycythemia vera myelofibrosis, and combinations thereof.

25. The method as claimed in claim 14, wherein the subject shows reduced level of bone marrow fibrosis or ameliorated inflammation after administering the composition.

26. The method as claimed in claim 25, wherein the subject further shows ameliorated splenomegaly or ameliorated anemia after administering the composition.


SEQUENCE LISTING (18 sequences)

SEQ ID NO: 1 - DNA, 20 bp, synthetic construct
  source 1..20: mol_type = other DNA; note = Forward primer COL1A1-F for PCR amplification of COL1A1 gene
tcgaggattgcccggaacag

SEQ ID NO: 2 - DNA, 24 bp, synthetic construct
  source 1..24: mol_type = other DNA; note = Reverse primer COL1A1-R for PCR amplification of COL1A1 gene
caccagtagcaccatcatttccac

SEQ ID NO: 3 - DNA, 20 bp, synthetic construct
  source 1..20: mol_type = other DNA; note = Forward primer FN1-F for PCR amplification of FN1 gene
cacctctgtgcagaccacat

SEQ ID NO: 4 - DNA, 20 bp, synthetic construct
  source 1..20: mol_type = other DNA; note = Reverse primer FN1-R for PCR amplification of FN1 gene
agttgccaccgtaagtctgg

SEQ ID NO: 5 - DNA, 20 bp, synthetic construct
  source 1..20: mol_type = other DNA; note = Forward primer ACTA2-F for PCR amplification of ACTA2 gene
agcgtggctattccttcgtt

SEQ ID NO: 6 - DNA, 20 bp, synthetic construct
  source 1..20: mol_type = other DNA; note = Reverse primer ACTA2-R for PCR amplification of ACTA2 gene
gcccatcaggcaactcgtaa

SEQ ID NO: 7 - DNA, 20 bp, synthetic construct
  source 1..20: mol_type = other DNA; note = Forward primer IL-1beta-F for PCR amplification of IL-1beta gene
gaccaccactacagcaaggg

SEQ ID NO: 8 - DNA, 20 bp, synthetic construct
  source 1..20: mol_type = other DNA; note = Reverse primer IL-1beta-R for PCR amplification of IL-1beta gene
atcgtgcacataagcctcgt

SEQ ID NO: 9 - DNA, 20 bp, synthetic construct
  source 1..20: mol_type = other DNA; note = Forward primer TNF-alpha-F for PCR amplification of TNF-alpha gene
cactttggagtgatcggccc

SEQ ID NO: 10 - DNA, 21 bp, synthetic construct
  source 1..21: mol_type = other DNA; note = Reverse primer TNF-alpha-R for PCR amplification of TNF-alpha gene
tcgtacaacatgggctacagg

SEQ ID NO: 11 - DNA, 20 bp, synthetic construct
  source 1..20: mol_type = other DNA; note = Forward primer TGF-beta1-F for PCR amplification of TGF-beta1 gene
tggtggaaacccacaacgaa

SEQ ID NO: 12 - DNA, 20 bp, synthetic construct
  source 1..20: mol_type = other DNA; note = Reverse primer TGF-beta1-R for PCR amplification of TGF-beta1 gene
gagcaacacgggttcaggta

SEQ ID NO: 13 - DNA, 20 bp, synthetic construct
  source 1..20: mol_type = other DNA; note = Forward primer IL-6-F for PCR amplification of IL-6 gene
gacagccactcacctcttca

SEQ ID NO: 14 - DNA, 20 bp, synthetic construct
  source 1..20: mol_type = other DNA; note = Reverse primer IL-6-R for PCR amplification of IL-6 gene
agtgcctctttgctgctttc

SEQ ID NO: 15 - DNA, 20 bp, synthetic construct
  source 1..20: mol_type = other DNA; note = Forward primer PPIA-F for PCR amplification of PPIA gene
cgcgtctcctttgagctgtt

SEQ ID NO: 16 - DNA, 20 bp, synthetic construct
  source 1..20: mol_type = other DNA; note = Reverse primer PPIA-R for PCR amplification of PPIA gene
acttgccaccagtgccatta

SEQ ID NO: 17 - DNA, 20 bp, synthetic construct
  source 1..20: mol_type = other DNA; note = Forward primer IDO-F for PCR amplification of IDO gene
gcaagaacgggacactttgc

SEQ ID NO: 18 - DNA, 20 bp, synthetic construct
  source 1..20: mol_type = other DNA; note = Reverse primer IDO-R for PCR amplification of IDO gene
cgtctgatagctgggggttg